(19) United States
(12) Patent Application Publication
(10) Pub. No.: US 2019/0093136 A1
(43) Pub. Date: Mar. 28, 2019

(54) PROCESS FOR ENZYMATIC HYDROLYSIS FROM A MIXTURE OF PRE-TREATED SUBSTRATES OF DIFFERENT POROSITIES

(71) Applicant: IFP Energies nouvelles, Rueil-Malmaison Cedex (FR)

(72) Inventors: Federico BATTISTA, Lyon (FR); Melanie GOMEZ ALMENDROS, Lyon (FR)

(73) Assignee: IFP Energies nouvelles, Rueil-Malmaison Cedex (FR)

(21) Appl. No.: 16/144,469

(22) Filed: Sep. 27, 2018

(30) Foreign Application Priority Data
Sep. 28, 2017 (FR) 17/59.032

(51) Int. Cl. C12P 19/02; C12M 1/00; C12P 7/08; C12N 1/14

(52) U.S. Cl. CPC C12P 19/02; C12M 45/09; C12N 1/22; C12N 1/14; C12P 7/08

(57) ABSTRACT

The present invention relates to a process for enzymatic hydrolysis in which, under agitation, pre-treated lignocellulosic substrates are brought into contact with water and with enzymes such that the mixture has a content of dry matter of between 12 and 35% by weight, said process being characterised in that a mixture is used of at least two pre-treated lignocellulosic substrates with different porosities, at least one of the substrates being a substrate said to be of low porosity having a porosity of less than 60% of the volume and the other substrate a substrate said to be of high porosity having a porosity greater than or equal to 60% of the volume, and said substrate of low porosity being present in a quantity of at least 30% by weight in relation to the total weight of said mixture.

### FIELD OF THE INVENTION

The present invention relates to a process for enzymatic hydrolysis from a mixture of pre-treated lignocellulosic substrates of different porosities allowing the conversion of cellulose into glucose. The glucose can then be used in various further steps such as for example in a fermentation step for the production of alcohols, or for the production of intermediates for chemistry.

### PRIOR ART

The development of economically viable processes for upgrading lignocellulosic biomass is currently a “hot topic”. The increasing scarcity of fossil resources and competition with food supplies have resulted in a search for novel pathways to the production of biofuels and chemical intermediates.

Since the 1970s, the transformation of lignocellulosic biomass after hydrolysis of the constituent polysaccharides into sugars has been the subject of many studies.

Lignocellulosic biomass is characterised by a complex structure constituted by three principal polymers, cellulose, hemicelluloses and lignin, the proportions of which vary as a function of the species of lignocellulosic biomass. A typical but not limiting composition is as follows: the cellulose is in a quantity in the range 35% to 50%, the hemicelluloses, which are polysaccharides essentially constituted by pentoses and hexoses, are in a quantity in the range 20% to 30% and the lignins are in a quantity in the range 15% to 25% by weight. Degradation of the biomass proves to be difficult, since the polysaccharides of the plant wall (cellulose and hemicelluloses) are intimately associated with lignin, which provides the walls with rigidity.

Of these three polymers, cellulose is the principal source of sugars, as it is constituted by glucose; this latter is readily upgraded.

Conventionally, processes for upgrading biomass by a biochemical pathway comprise a plurality of steps. A first step is collection and transport of the lignocellulosic biomass to a biomass transformation centre. The second step is the pre-treatment or pre-hydrolysis of the biomass, which renders the cellulose accessible to the enzymes and thus capable of producing a pre-treated lignocellulosic substrate. The third step, enzymatic hydrolysis, means that, because a solution of cellulolytic and hemicellulolytic enzymes produced by microorganisms and known as an enzymatic cocktail is used, cellulose is transformed into glucose. This glucose may then be upgraded to intermediate products, for example to ethanol, during a fourth step of fermentation, generally by the yeast Saccharomyces cerevisiae, or to an acetone, butane, ethanol (ABE) mixture by fermentation by the yeast Clostridium acetobutylicum. A fifth step, distillation, then means that the molecules obtained can be concentrated. The glucose can also be upgraded to biofuels (hydrogen, methane).

One of the key steps is thus the enzymatic hydrolysis. In the enzymatic hydrolysis step, said pre-treated lignocellulosic substrate must be mixed with a liquid solution containing the cellulolytic and hemicellulolytic enzymes. Since the aim is to obtain a high concentration of sugars, the enzymatic hydrolysis step must be carried out at high concentrations of pre-treated lignocellulosic substrate, that is to say at a high content of dry matter. It has been estimated that the process is economically viable when a minimum concentration of sugars of 8% by weight is produced during the enzymatic hydrolysis, which corresponds to a content of dry matter of approximately 15% by weight (McIntosh, S., Zhang, Z., Palmer, J., Wong, H., Doherty, W. O. S., Vancov, T., 2016. Pilot-scale cellulosic ethanol production using eucalyptus biomass pre-treated by dilute acid and steam explosion. Biofuels, bioproducts and biorefining 10 (4), 346-358). Working at a high content of dry matter also allows a reduction in the volume of the reactor and, as a consequence, a reduction in the financial and energy costs of the process (Larsen, J., Ostergaard Petersen, M., Thirup, L., Wen Li, H., Krogh Iversen, F., 2008. The IBUS process of lignocellulosic bioethanol close to a commercial reality. Chem. Eng. Technol. 31, 765-722).

However, intimate mixing of the pre-treated lignocellulosic substrate with said liquid solution containing the cellulolytic and hemicellulolytic enzymes can prove difficult when the contents of dry matter are high. In fact, the start of the enzymatic hydrolysis at a high content of dry matter poses particular problems of mixing and homogenisation. The reaction medium is very pasty and viscous which calls for special agitation that is much more complex than that necessary at the end of hydrolysis when the reaction mixture has become more liquid.

The problems of viscosity associated with a high content of dry matter are known. Cara et al. (Cara, C., Moya, M., Ballesteros, I., Negro, M. J., González, A., Ruiz, E., 2007. Influence of solid loading on enzymatic hydrolysis of steam exploded or liquid hot water pretreated olive tree biomass. Process Biochemistry, 42, 1003-1009) and Battista et al. (Battista, F., Fino, D., Mancini, G., Ruggeri, B., 2016. Mixing in digesters used to treat high viscosity substrates: The case of olive oil production wastes. Journal of Environmental Chemical Engineering 4, 915-923) have shown that when the content of dry matter is high, the complexity of the lignocellulosic polymers causes an increase in viscosity of the reaction medium and consequently poor mixing within the bioreactor.

Moreover, it has been shown that there is a link between the viscosity and the porosity of the pre-treated lignocellulosic biomass. Enzymatic hydrolysis when there is a high content of dry matter is very difficult when the lignocellulosic substrate is characterised by a high degree of porosity (Lewandowska, M., Szymanka, K., Kordala, N., Dabrowska, A., Bednarski, W., Juszczuk, A., 2016. Evaluation of mucor indicus and Saccharomyces cerevisiae capability to ferment hydrolysates of rape straw and Miscanthus giganteus as affected by the pretreatment method. Bioresource Technology 212, 262-270). A high porosity of the pre-treated lignocellulosic substrate causes a high degree of impregnation of water. A high degree of water impregnation reduces the amount of liquid in the reaction medium and as a consequence causes an increase in the viscosity due to lower dispersion and homogenisation of the substrate in the reactor.

To overcome this problem of viscosity when working at a high content of dry matter, during its research the applicant has developed a new process for enzymatic hydrolysis using a mixture of at least two lignocellulosic substrates of different porosities, one having a high porosity, the other having a low porosity, with the two substrates being used in a certain ratio.

More specifically, the present invention relates to a process for enzymatic hydrolysis in which, under agitation, pre-treated lignocellulosic substrates are brought into contact with water and with enzymes such that the mixture has a content of dry matter of between 12 and 35% by weight, said process being characterised in that a mixture is used of at least two pre-treated lignocellulosic substrates with different porosities, at least one of the substrates being a substrate said to be of low porosity having a porosity of less than 60% of the volume and the other substrate a substrate said to be of high porosity having a porosity greater than or equal to 60% of the volume, and said substrate of low porosity being present in a quantity of at least 30% by weight in relation to the total weight of said mixture.

The applicant is consequently proposing a process for enzymatic hydrolysis from a mixture of substrates of different porosities. In this way, it is possible to work with a high content of dry matter without having the rheological problems due to water impregnation of the high porosity substrates. In fact, as previously indicated the physical properties of the substrates influence the viscosity of the reaction medium. The substrates of high porosity can assimilate more water from the reaction medium and thereby cause the increase in viscosity. Conversely, the substrates of low porosity do not cause these problems of viscosity. By mixing a substrate of high porosity with a substrate of low porosity it is therefore possible to achieve an improvement in mixing through the drop in viscosity.

However, the applicant has noticed that the use of the two substrates in a certain ratio allows a synergy effect to be seen in terms of the drop in viscosity. In fact, the effect of impregnation of water by substrates of high porosity is cancelled out by the substrates of low porosity when the concentration of the substrates of low porosity is at least at least 30% by weight in said mixture.

An advantage of the present invention is that it provides a process for enzymatic hydrolysis in which, thanks to the reduction in viscosity, the mixing time is reduced.

Another advantage of the present invention is to provide a process for enzymatic hydrolysis in which it is possible to work with lower reactor volumes.

Moreover, another advantage of the present invention is to provide a process for enzymatic hydrolysis in which a reduction in energy consumption is observed.

Furthermore, the possibility of working at a high content of dry matter with substrates that are often treated separately allows a reduction in the number and volume of the reactors.

Another advantage of the present invention is to provide a process for enzymatic hydrolysis allowing monitoring of, and simple adaptation to, changes in the reaction medium without the need for complex measurements.

According to a variant, the substrate of low porosity is present in a quantity of between 30 and 50% by weight, and preferably of between 40 and 50% by weight in relation to the total weight of said mixture.

According to a variant, the substrate said to be of low porosity has a porosity of less than 58% of the volume.

According to a variant, the substrate said to be of low porosity has an apparent density of greater than 680 kg/m3.

According to a variant, the substrate said to be of low porosity is miscanthus.

According to a variant, the substrate said to be of high porosity has a porosity of greater than 65% of the volume.

According to a variant, the substrate said to be of high porosity has an apparent density of between 530 and 680 kg/m³.

According to a variant, the substrate said to be of high porosity is wheat straw.

According to a variant, the pre-treated lignocellulosic substrates are brought into contact at a content of dry matter of between 18 and 24% by weight.

According to a variant, the process takes place at a temperature of between 40 and 60° C., at a pH of between 4 and 6, and at atmospheric pressure.

According to a variant, said process is implemented in a sequentially fed reactor during which no racking of the contents of the reactor is carried out.

According to another variant, said process is implemented in a batch reactor.

According to a variant, said process is followed by a fermentation step in the presence of an alcohol-producing microorganism.

According to another variant, said process is carried out in the presence of an alcohol-producing microorganism according to a process of simultaneous saccharification and fermentation known as a SSF process.

### DETAILED DESCRIPTION OF THE INVENTION

The pre-treated lignocellulosic biomass is obtained from wood (deciduous and resinous), raw or treated, by-products of agriculture such as straw, plant fibres, forestry crops, alcohol-, sugar- and cereal-producing plant residues, resides from the paper industry, marine biomass (such as macroalgae cellulosic residue) or lignocellulosic material conversion products.

The lignocellulosic substrates used in the process of the invention are the result of pre-treating the biomass under conditions that allow a destructuring of the lignocellulose by modifying the physical and physico-chemical properties of the lignocellulosic material. The pre-treatment step can be carried out using any of the types of pre-treatment of lignocellulosic biomass known to the person skilled in the art. A pre-conditioning step including, by way of example, crushing or stone-removal, may also be carried out. The pre-treatment step may involve heat, chemical, mechanical and/or enzymatic treatment or a combination of these treatments.

According to a preferred variant, the pre-treatment step is selected from among pre- treatment under acid conditions such as acid cooking or steam explosion under acid conditions, pre-treatment in alkaline media such as pre-treatment with sodium sulphide (Kraft process), an ammonia recycle percolation process (ARP) or an ammonia fibre explosion process (AFEX), oxidising pre-treatment such as pre-treatment with ozone, hydrogen peroxide, oxygen or peracetic acid, pre-treatment without the addition of chemical reagents such as steam explosion without addition of acid or pre-treatment by washing with very hot water, or also an organosolv process.

The pre-treatment step is advantageously a pre-treatment by steam explosion under acid conditions, preferably under optimum conditions of 150 to 250° C. for a few minutes.

The mixture of at least two pre-treated lignocellulosic substrates of different porosities used in the process of the invention comprises at least one substrate said to be of low porosity having a porosity of less than 60% of the volume and another substrate said to be of high porosity having a porosity greater than or equal to 60% of the volume. The degree of porosity is measured by the nitrogen sorption and desorption isotherms method (Horvath, G., Kawazoe, K., 1983. Method for calculation of effective pore size distribution in molecular sieve carbon, J. Chem. Eng. Jpn. 16, 470).

The substrate said to be of low porosity has a porosity of less than 60% of the volume, and preferably of less than 58% of the volume. Generally, the substrate said to be of low porosity has a porosity of between 45 and less than 60% of the volume, and preferably between 48 and 58 of the volume.

The substrate said to be of low porosity is generally characterised by an apparent density of greater than 680 kg/m³. The apparent density is measured using the Archimedes principle.

The substrate said to be of low porosity is advantageously miscanthus. Miscanthus generally has a porosity of between 49 and 55% of the volume. Miscanthus generally has an apparent density of between 700 and 750 kg/m³.

The substrate said to be of high porosity has a porosity greater than or equal to 60% of the volume, preferably greater than 65% of the volume. Generally, the substrate said to be of high porosity has a porosity of between 60 and 80% of the volume, preferably between 65 and 79% of the volume.

The substrate said to be of high porosity is generally characterised by an apparent density of between 530 and 680 kg/m³, and preferably between 550 and 670 kg/m³.

The substrate said to be of high porosity is advantageously wheat straw. Wheat straw generally has a porosity of between 69 and 77% of the volume. Wheat straw generally has an apparent density of between 570 and 650 kg/m³.

Said substrate of low porosity is present in a quantity of at least 30% by weight, preferably at least 40% by weight, in relation to the total weight of said mixture.

Said substrate of low porosity is preferably present in a quantity of at the most 50% by weight in relation to the total weight of said mixture.

Preferably, the substrate of low porosity is present in a quantity of between 30 and 50% by weight, preferably of between 40 and 50% by weight, in relation to the total weight of said mixture.

Throughout the remainder of the text, the concentration of pre-treated lignocellulosic substrate is expressed as a percentage by weight of dry matter. The content of dry matter is measured according to standard ASTM E1756-08(2015) “Standard Test Method for Determination of Total Solids in Biomass”.

According to the invention, the pre-treated lignocellulosic substrates are brought into contact in the process of the present invention with water and with enzymes such that the mixture has a content of dry matter of between 12 and 35% by weight, preferably between 15 and 30% by weight, and most preferably between 18 and 24% by weight.

According to the invention, the enzymes are brought into contact in the process according to the present invention with a concentration of between 0.1 and 60 mg of enzymes per gram of cellulose, preferably a concentration of between 5 and 30 mg of enzymes per gram of cellulose and most preferably of between 10 and 20 mg of enzymes per gram of cellulose.

The enzymatic hydrolysis is generally carried out at a pH of between 4 and 6, preferably between 4.5 and 5.8 and more preferably again between 4.8 and 5.5. It generally takes place at a temperature of between 40 and 60 ° C., and preferably between 50 and 55° C. It advantageously takes place at atmospheric pressure.

The enzymatic hydrolysis is carried out by means of enzymes produced by a microorganism. The enzymatic solution added contains enzymes that break down the cellulose into sugars. Microorganisms, such as fungi of the genus Trichoderma, Aspergillus, Penicillium or Schizophyllum, or anaerobic bacteria of, for example, the genus Clostridium, produce these enzymes, which in particular contain cellulases suited to the extensive hydrolysis of the cellulose. In a highly preferred manner, the cellulolytic enzymes of step d) are produced by the microorganism Trichoderma reesei.

According to the invention the period of contact at the time of the enzymatic hydrolysis is between 1 and 200 hours, preferably between 2 and 120 hours, and most preferably between 24 and 120 hours.

The process of the invention can be carried out in continuous or discontinuous mode (also known as batch mode), or with sequential feeding (also known as fed-batch mode), in one or more reactors. According to a variant, the process of the invention is carried out discontinuously in a closed reactor, also known as a batch reactor.

Said process according to the present invention can be monitored by measuring over time the value of one of the rheological characteristics of the reaction medium which are advantageously selected from among the viscosity of the reaction medium, the torque of the shaft of the agitation system and the electrical power consumed by the motor. The electrical power consumed by the motor has the notation P_{elec}.

During the process of the invention, the viscosity of the reaction medium, the torque of shaft of the agitation system and the electrical power consumed by the motor are rheological characteristics that are of interest from a number of aspects for monitoring the lignocellulosic substrate produced. In fact, these characteristics of viscosity, torque and power are inter-related. The electrical power consumed by the motor P_{elec} is linked to the mechanical power P_{mech} driving the stirrer shaft.

The electrical power consumed by the motor is a parameter that is conventionally measured and monitored in pilot or industrial installations.

The following formulas define the relationships between the various parameters:

P_{mech}=f (P_{elec}), f being a design characteristic of the motor which is specified by the motor constructor.

P_{mech}=2πN*C in which:

N is the speed of agitation in revolutions per second,

C is the torque in N·m,

and P_{mech} is the power in Watts.

During agitation the following relationship applies:

P_{mech}=ρNₚN³D⁵

ρ is the density of the reaction medium in kg·m⁻³

D is the diameter of the stirrer in m,

Nₚ is a characteristic of the stirrer that depends on the geometry of the tank and the flow regime.

During a laminar flow regime, the following relationship applies:

Nₚ=A/Re, hence P_{mech}=ρAN³D⁵/Re

with A being a constant of the agitation system and Re the Reynolds number with Re=ρND²/μ̅, μ̅ is the mean dynamic viscosity measured in Pascal seconds (Pa·s) of the reaction medium with μ̅=P_{mech}/(AN²D³)=2πC/(AD³N)

While the viscosity and torque of the shaft of the agitation system are measurements that are easily accessible on a small scale, the electrical power consumed by the motor P_{elec} is the magnitude most easily measurable on an industrial scale.

In a highly preferred manner, said process according to the present invention is characterised in that a measurement is performed over time of the electrical power consumed by the motor.

Said process according to the present invention is advantageously carried out in a reactor, preferably with a cylindrical shape, with a height/diameter ratio which is advantageously in the range 1 to 3.

Said reactor allows the processing of viscous media with variable viscosity and thus the application of dry matter content of lignocellulosic substrate that can reach 35% by weight. The high contents of dry matter and the high viscosity of the reaction medium mean that the reactor must be fitted with a stirrer allowing good contact between enzymes and substrate and good homogeneity. Conventionally, the stirrer selected must be capable of processing laminar flows. Wide stirrers, or even those which scrape the wall of the reactor at moderate speeds of rotation and applying a blending and kneading action are preferred. An example of a particularly suitable stirrer is the Paravisc® (EKATO) which allows the addition of a counter-paddle to break up combined movements.

The speed of agitation depends on the size of the reactor and of the stirrer.

In the event of measuring the electrical power consumed over time by the motor, said electrical power consumed by the motor in relation to the mass of the reaction volume advantageously remains between 0.05 and 4 kW/tonne and preferably between 0.5 et 2 kW/tonne.

According to a preferred embodiment, the process of enzymatic hydrolysis according to the invention can be followed by a step of alcoholic fermentation by an alcohol-producing microorganism in order to produce a fermented effluent containing alcohol.

The enzymatic hydrolysis and the alcoholic fermentation can also be performed simultaneously. It is a case here of a simultaneous saccharification and fermentation or SSF process. The enzymatic hydrolysis and the alcoholic fermentation can also be implemented according to other arrangements known to the person skilled in the art, such as the Presacchararification followed by Simultaneous Saccharification and Fermentation process (PSSF) or also the Hybrid Hydrolysis and Fermentation process (HHF).

The sugars obtained by enzymatic hydrolysis can be fermented into alcohols such as ethanol, 1,3-propanediol, isopropanol, 1-butanol, isobutanol or 1,4-butanediol, on their own or as a mixture. The alcoholic fermentation preferably produces ethanol.

The alcoholic fermentation is ensured by fungi or other alcohol-producing microorganisms. Within the meaning of this invention, the term “alcoholic fermentation” designates a process of fermentation of the sugars into alcohol (s) by means of microorganisms alone. The alcohol-producing microorganisms used during the alcoholic fermentation step of the hexoses are preferably selected from among fungi and bacteria, which may have been genetically modified.

When the alcohol-producing microorganism is a yeast, Saccharomyces cerevisiae is the most effective. It is also possible to select yeasts such as Schizosaccharomyces pombe or Saccharomyces uvarum or diastaticus. More thermophilic yeasts, such as Kluyveromyces fragilis (now often designated by K. marxianus) are also of interest, particularly when the enzymatic hydrolysis and the alcoholic fermentation are performed simultaneously (SSF process).

A genetically modified organism, such as for example a yeast of the Saccharomyces cerevisiae type such as TMB 3400 (Ohgren et al, J. of Biotech 126, 488-498, 2006) may also be used.

When the alcohol-producing microorganism is a bacterium, preference will be for Zymomonas mobilis which offers an effective means of assimilation for the production of ethanol, or anaerobic bacteria of the genus Clostridium, such as for example Clostridium acetobutylicum for the production of mixtures of alcohols and solvents such as acetone-butanol-ethanol (ABE) or isopropanol-butanol-ethanol (IBE), or also Escherichia coli for the production of isobutanol, for example.

The alcoholic fermentation is preferentially carried out at a temperature of between 30° C. and 40° C., and a pH of between 3 and 6.5.

Yeasts, and preferably Saccharomyces cerevisiae are the highly-preferred microorganisms used. They have greater robustness and safety and do not require sterile conditions to operate the process and plant.

Yeasts of the genus Saccharomyces are capable of fermenting solely hexoses (essentially glucose and mannose). These yeasts upgrade hexoses into ethanol in an optimum manner and allow good conversion yields to be obtained.

When the enzymatic hydrolysis and the alcoholic fermentation are carried out in the same operation (SSF process), the temperature is preferably between 30 and 45° C., and the pH between 4 and 6 in order to stimulate the performance of the yeasts.

The operational example below is intended to illustrate the invention.

### BRIEF DESCRIPTION OF THE DRAWINGS

FIG. 1 represents the results of the enzymatic hydrolysis tests of the Examples.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 17/59.032, filed Sep. 28, 2017, are incorporated by reference herein.

### EXAMPLES

The substrates used for the test examples are pre-treated wheat straw and miscanthus, respectively. Table 1 summarises the physical and chemical characteristics of the pre-treated wheat straw and miscanthus. The pre-treated substrates generally have a dry matter of between 40 and 60% by weight, said dry matter in the table referring to the pre-treated substrate as is. Batch tests on a substrate of high porosity (wheat straw), on a substrate of low porosity (miscanthus) and on a combination of the two at different ratios were carried out (Table 2). All the tests were performed at a content of dry matter of 20% by weight in the mixture of the process.

The performances in the tests were assessed taking into account the three factors of: (i) mixing time, (ii) energy consumption by the mixture and (iii) the concentration of glucose contained in the reaction mixture at the end of the enzymatic hydrolysis.

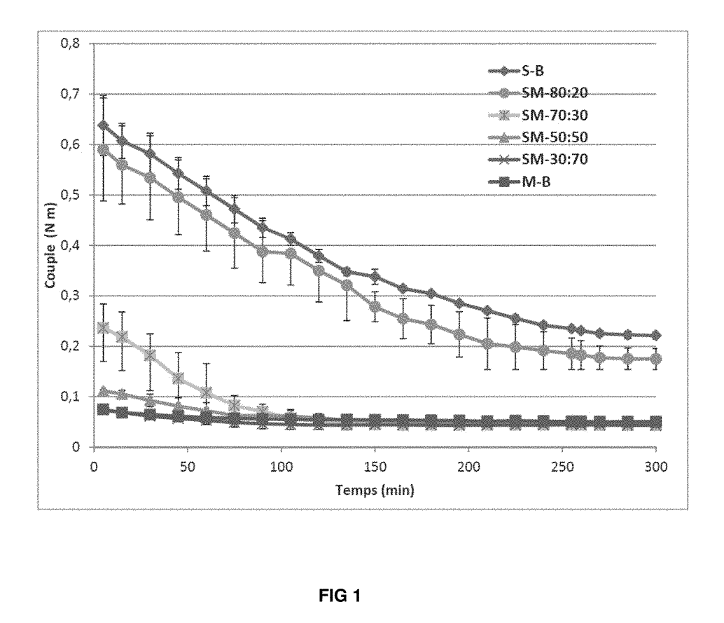

The results show that the rheological behaviour during the enzymatic hydrolysis depends on the substrates used to feed the reactor (FIG. 1): the torque is higher for wheat straw than for miscanthus. Wheat straw (test S-B) recorded a considerable reduction in the torque values between the start and the end of the tests. In fact, the torque required for a good mixing of the reaction medium drops from 0.64 Nm to 0.22 Nm for the S-B test. Furthermore, the torque in the case of the miscanthus test (test M-B) drops from 0.09 to 0.045 Nm (FIG. 1).

The value of the torque is closely correlated to the energy consumption for the mixing. As a consequence, test S-B required more than 32.5 kJ, whereas test M-B required between 5 and 7 kJ, with an energy reduction of nearly 80%. The mixing time, which is an indicator of the rheological performances in the reactor, was more than 50 s for the wheat straw compared to just 17 s for the miscanthus.

The concentration of glucose was similar for all the tests: between 19 and 21 g/L.

The differing rheological and energy values for the two substrates can be explained by the viscosity. The reaction medium composed of 20% by weight of dry matter of wheat straw (test S-B) was highly viscous, with an apparent viscosity of 420.1±3.1 cP. Conversely, the miscanthus (test M-B) had a gentle fluid-dynamic behaviour with low viscosity values 79.40±4.05 cP at 20% by weight of dry matter. This difference is due to a different physical structure of the two substrates. Table 1 shows that the wheat straw has a higher degree of porosity than the miscanthus (73% of the volume and 52% of the volume, respectively). The rheological behaviour can then be explained by an impregnation of water that is much higher for the particles with greater porosity (wheat straw).

Moreover, batch tests (Table 2) were carried out on mixtures of wheat straw and miscanthus at different concentrations. As expected, the reduction in the concentration of wheat straw in the mixture allowed a reduction of the mixing time and the energy consumptions. In test SM-80:20, the viscosity was still highly influenced by the presence of wheat straw. However, when the content of miscanthus present in the reaction mixture reaches 30% by weight (test SM-70:30), the viscosity of the mixture falls and its behaviour is similar to that observed during test M-B.

The reduction in torque values as a result of the increase in the miscanthus content in the reaction mixture is of major significance for the energy consumption. Test SM-80:20 required 30.5 kJ for the mixing, similar to test S-B, but the total energy consumption falls to around 8 kJ for test SM-70:30. This value is very close to the 5.8 kJ recorded with test M-B. A synergy effect can therefore be seen in terms of the fall in viscosity. In fact, the effect of the water impregnation by the substrates of high porosity is cancelled out by the substrates of low porosity when the concentration of substrates of low porosity is at least 30% by weight in said mixture.

TABLE 1
Description of the wheat straw and miscanthus

| | Miscanthus | Wheat straw |
|---|---|---|
| Dry matter of the pre-treated substrate | 47.73 ± 144 | 46.06 ± 1.90 |
| (% by weight) | | |
| Apparent density (kg/m³) | 722.54 ± 12.14 | 607.46 ± 18.76 |
| Porosity (% of the volume) | 52.00 ± 2.60 | 73.00 ± 3.65 |

TABLE 2
Abbreviations and description of the tests.

| Abbreviation | Description of the tests |
|---|---|
| S-B | Batch test with wheat straw |
| M-B | Batch test with miscanthus |
| SM-80:20 | Batch test with a mixture composed of 80% by weight of |
| | wheat straw and 20% by weight of miscanthus |
| SM-70:30 | Batch test with a mixture composed of 70% by weight of |
| | wheat straw and 30% by weight of miscanthus |
| SM-50:50 | Batch test with a mixture composed of 50% by weight of |
| | wheat straw and 50% by weight of miscanthus |
| SM-30:70 | Batch test of a mixture composed of 30% by weight of |
| | wheat straw and 70% by weight of miscanthus |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for enzymatic hydrolysis in which, under agitation, pre-treated lignocellulosic substrates are brought into contact with water and with enzymes such that the mixture has a content of dry matter of between 12 and 35% by weight, said process being characterised in that a mixture is used of at least two pre-treated lignocellulosic substrates with different porosities, at least one of the substrates being a substrate said to be of low porosity having a porosity of less than 60% of the volume and the other substrate a substrate said to be of high porosity having a porosity greater than or equal to 60% of the volume, and said substrate of low porosity being present in a quantity of at least 30% by weight in relation to the total weight of said mixture.

2. Process according to claim 1 in which the substrate of low porosity is present in a quantity of between 30 and 50% by weight in relation to the total weight of said mixture.

3. Process according to claim 1 in which the substrate of low porosity is present in a quantity of between 40 and 50% by weight in relation to the total weight of said mixture.

4. Process according to claim 1 in which the substrate said to be of low porosity has a porosity of less than 58% of the volume.

5. Process according to claim 1 in which the substrate said to be of low porosity has an apparent density of greater than 680 kg/m3.

6. Process according to claim 1 in which the substrate said to be of low porosity is miscanthus.

7. Process according to claim 1 in which the substrate said to be of high porosity has a porosity of greater than 65% of the volume.

8. Process according to claim 1 in which the substrate said to be of high porosity has an apparent density of between 530 and 680 kg/m³.

9. Process according to claim 1 in which the substrate said to be of high porosity is wheat straw.

10. Process according to claim 1 in which the pre-treated lignocellulosic substrates are brought into contact at a content of dry matter of between 18 and 24% by weight.

11. Process according to claim 1 in which the process takes place at a temperature of between 40 and 60° C., at a pH of between 4 and 6, and at atmospheric pressure.

12. Process according to claim 1 in which said process is implemented in a sequentially fed reactor during which no racking of the contents of the reactor is carried out.

13. Process according to claim 1 in which said process is implemented in a batch reactor.

14. Process according to claim 1 in which said process is followed by a fermentation step in the presence of an alcohol-producing microorganism.

15. Process according to claim 1 in which said process is carried out in the presence of an alcohol-producing microorganism according to a process of simultaneous saccharification and fermentation.
